(19) United States
(12) Patent Application Publication
(10) Pub. No.: US 2008/0071118 A1
(43) Pub. Date: Mar. 20, 2008

(54) Process for producing polyether polyol

(76) Inventor: Naoko Fujita, (US)

Correspondence Address: OBLON, SPIVAK, MCCLELLAND MAIER & NEUSTADT, P.C., 1940 DUKE STREET, ALEXANDRIA, VA, 22314, US

(21) Appl. No.: 11/631,015

(22) PCT Filed: Jun. 29, 2005
(86) PCT No.: PCT/JP05/11980

§ 371 Date: Jun. 1, 2007

(30) Foreign Application Priority Data
Jun. 29, 2004 (JP) 2004-191567
Jun. 29, 2004 (JP) 2004-191568
Aug. 23, 2004 (JP) 2004-242744

(51) Int. Cl. C07C 41/03

(52) U.S. Cl. 568/680

(57) ABSTRACT

To provide a method of producing a polyether polyol having less coloration with good selectivity and high efficiency by dehydrocondensing a polyol. In producing a polyether polyol by dehydrocondensation reaction of a polyol, a solid acid catalyst satisfying at least one of the following requirements (1) to (3) is used:
(1) Acid function H₀ measured by Hammett's indicator adsorption method is larger than −3; (2) In Temperature-Programmed Desorption (TPD) analysis of ammonia, desorption amount of ammonia in a region of from 100 to 350° C. is 60% or more of the entire ammonia desorption amount (a region of from 25 to 700° C.); and (3) In thermogravimetry (TG), desorption amount of water is 3% by weight or more of a reference weight in a region of from 32 to 250° C.

### TECHNICAL FIELD

The present invention relates to a method of producing a polyether polyol by dehydrocondensation reaction of a polyol. More particularly, it relates to a method of efficiently producing a polyether polyol having less coloration by conducting this reaction in the presence of a catalyst having specific acid properties.

### BACKGROUND ART

Polyether polyol is a polymer having wide applications including a raw material for a soft segment of elastic fibers, thermoplastic elastomer and the like. Polyethylene glycol, poly(1,2-propanediol), polytetramethylene ether glycol, and the like are known as the representative examples of the polyether polyol. Of those, the poly(1,2-propanediol) is liquid at room temperature, thereby being easy to handle, and is inexpensive. As a result, it is widely used. However, the poly(1,2-propanediol) has a primary hydroxyl group and a secondary hydroxyl group, and difference in physical properties of those hydroxyl groups becomes problem depending on the application. Contrary to this, polytrimethylene ether glycol which is a dehydrocondensate of 1,3-propanediol has only primary hydroxyl group, and further has low melting point. Therefore, it is recently noted.

The polyether polyol can generally be produced by dehydrocondensation reaction of the corresponding polyol. However, ethylene glycol 1,4-butanediol and 1,5-pentanediol produce 5-membered or 6-membered cyclic ethers, that is, 1,4-dioxane, tetrahydrofuran and tetrahydropyran, respectively when subjected to dehydrocondensation. For this reason, polyether polyol corresponding to a polymer of ethylene glycol or 1,4-butanediol is produced by ring opening polymerization of the corresponding cyclic ether, that is, ethylene oxide or tetrahydrofuran. Polyether polyol corresponding to a polymer of 1,5-pentanediol is that tetrahydropyran which is a cyclic ether is thermodynamically predominant, and therefore, its production is difficult.

Production of a polyether polyol by dehydrocondensation reaction of a polyol is generally conducted using an acid catalyst. Iodine, an inorganic acid such as hydrogen iodide or sulfuric acid, an organic acid such as p-toluenesulfonic acid (see Patent Document 1), a resin having a perfluoroalkylsulfonic acid group at a side chain (see Patent Document 2), a combination of sulfuric acid and cuprous chloride, activated clay, zeolite, an organosulfonic acid, a heteropolyacid (see Patent Document 3) and the like are proposed as the catalyst.

A method of conducting dehydrocondensation reaction under nitrogen atmosphere and then conducting dehydrocondensation reaction under reduced pressure (see Patent Document 4) is proposed as the reaction method.

Of those, iodine, an inorganic acid such as hydrogen iodide or sulfuric acid, an organic acid such as p-toluenesulfonic acid, an organosulfonic acid, heteropolyacid and the like are a homogeneous acid catalyst. Where the homogeneous acid catalyst is used, the acid catalyst shows strong acid properties. Therefore, there were the problems that a reactor used in polymerization reaction corrodes, by the corrosion of the reactor, metal components elute to color a polyether polyol as a product, and the metal components eluted are contained in the polyether polyol. Further, to prevent corrosion, it is necessary to employ a glass reactor or a glass-lined reaction, or to use a reactor using a high class material such as hastelloy, and this gave rise great problem in the case of constructing large-scaled facilities and in the point of construction cost. Additionally, where the homogeneous catalyst is used, there is the case that an ester originated from the acid of the catalyst is contained in the terminal of a polyether polyol as a product, requiring hydrolysis of the ester. This gives rise to the problems on an increased number of steps and waste water treatment. Further, because the homogeneous acid catalyst is contained in the polyether polyol, those acid catalysts must be removed by methods such as neutralization and water washing, and there was the problem of requiring purification step of the polyether polyol for the removal.

If a solid catalyst can be used as the catalyst, it can solve all of the problems, and such is a markedly advantageous method.

Examples of the solid acid catalyst that can be used in dehydrocondensation reaction of a polyol include a resin having a perfluoroalkylsulfonic acid group at a side chain, activated clay and zeolite as described in the above Patent Documents. However, where those are used in dehydrocondensation reaction of a polyol, there are the problems that side reaction products such as allyl alcohol are produced in large amount, polyether polyol selectivity is very low, and the polyether polyol itself is colored vigorously. Thus, those were not in a level that can be put into practice.

Patent Document 1: U.S. Pat. No. 2,520,733

Patent Document 2: International Publication 92/09647, pamphlet

Patent Document 3: U.S. Pat. No. 5,659,089

Patent Document 4: US-A-2002/0007043

### DISCLOSURE OF THE INVENTION

### Problems that the Invention is to Solve

The present invention provides a method of producing a polyether polyol having less coloration with good selectivity and high efficiency by dehydrocondensing a polyol using a solid catalyst.

### Means for Solving the Problems

Because strong acid is active in the case of the homogeneous catalyst as described above, it was considered in the present reaction that a solid catalyst having strong acid properties is preferable even in the case of a heterogeneous catalyst. However, surprisingly, from the investigations by the present inventors it was clarified that too strong acid point accelerates a side reaction such as intramolecular dehydration, and further clarified that a catalyst having strong acid properties is unsuitable. Accordingly, the present inventors have found that the above object can be achieved by using a solid acid catalyst that does not have strong acid point, and have reached to complete the present invention.

That is, the object of the present invention resides in a method of producing a polyether polyol, characterized by in that in producing a polyether polyol by dehydrocondensation reaction of a polyol, a solid acid catalyst satisfying at least one of the following requirements (1) to (3) is used.

Acid function H₀ measured by Hammett's indicator adsorption method is larger than −3.

In Temperature-Programmed Desorption (TPD) analysis of ammonia, desorption amount of ammonia in a region of from 100 to 350° C. is 60% or more of the entire ammonia desorption amount (a region of from 25 to 700° C.).

In thermogravimetry (TG), desorption amount of water is 3% by weight or more of a reference weight in a region of from 32 to 250° C.

A second object resides in the method of producing a polyether polyol as described above, wherein the solid acid catalyst contains a metal element and/or an organic base in an amount of from 0.01 to 2.5 equivalents to the acid.

A third object resides in the method of producing a polyether polyol as described above, wherein the metal element is an alkali metal.

A fourth object resides in the method of producing a polyether polyol as described above, wherein the organic base has a pyridine skeleton.

A fifth object resides in the method of producing a polyether polyol as described above, wherein the solid acid catalyst, and a metal element-containing compound and/or the organic base are used in combination.

A sixth object resides in the method of producing a polyether polyol as described above, wherein the solid acid catalyst is at least one selected from the group consisting of an intercalation compound, zeolite, a mesoporous substance, a metal composite oxide, an oxide or a composite oxide containing a sulfonic acid group, a carbon material containing a sulfonic acid group, and a resin having a perfluoroalkylsulfonic acid group at a side chain.

A seventh object resides in the method of producing a polyether polyol as described above, wherein the polyol is a diol of from 3 to 10 carbon atoms having two primary hydroxyl groups (excluding a diol that forms a 5-membered or 6-membered cyclic ether by dehydration), or a mixture of the diol and other polyol, wherein the proportion of the other polyol is less than 50 mol %.

An eighth object resides in the method of producing a polyether polyol as described above, wherein the reaction is conducted at a temperature of from 120 to 250° C.

### ADVANTAGE OF THE INVENTION

According to the production method of the present invention, a polyether polyol having less coloration can efficiently be produced under mild conditions.

### BEST MODE FOR CARRYING OUT THE INVENTION

<Solid Acid Catalyst>

The solid acid catalyst used in the production method of the present invention is satisfied with at least one of the requirements (1) to (3) described below. Of those requirements, the catalyst satisfying two requirements is more preferable. Specifically, the solid acid catalyst satisfying (1) and (2), (1) and (3), or (2) and (3) is more preferable. The solid acid catalyst satisfying all of the requirements (1), (2) and (3) is particularly preferable.

(1) Hammett's Indicator Adsorption Method

The solid acid catalyst used in the present invention requires that acid strength is not too strong in the present reaction, and has acid function H₀ measured with Hammett's indicator adsorption method of −3 or larger, preferably +1.5 or larger, and particularly preferably +2 or larger. The acid function H₀ measured with Hammett's indicator adsorption method has the strong acid point with decreasing its numerical value. Therefore, the acid function H₀ having −3 or larger means weak acid properties that do not involve coloration of acidic color by the Hammett's indicator of pKa=−3.

Hammett's acid strength function used herein is obtained by treating a solid acid catalyst in a saturated water vapor at 25° C. for 2 days, and measuring acid strength with Hammett's indicator in a commercially available benzene solution.

In Hammett's indicator method, in the case of showing acidity, an indictor for measuring acidity can easily measure by that the Hammett's indicator changes in acid color on a solid acid catalyst. In the case that the solid acid catalyst is originally colored, it is difficult to visually recognize color change of the indicator. In such a case, change of the indicator is analyzed by, for example, spectroscopic method.

(2) Temperature Programmed Desorption Method (TPD) of Ammonia

Acid content and acid strength of the so-lid acid catalyst used in the present reaction is preferable that the amount of acid point is not large. It is preferable that ammonia desorption amount in a region of from 100 to 350° C. by temperature programmed desorption (TDS: Temperature Programmed Desorption) of ammonia is 60% or more of the entire ammonia desorption amount (a region of from 25 to 700° C.). Above all, it is more preferable that the ammonia desorption amount is 70% or more.

Further, it is preferable that ammonia desorption amount in a region of from 100 to 300° C. is 50% or more of the entire ammonia desorption amount (a region of from 25 to 700° C.) and it is more preferable to be 60% or more.

Furthermore, it is preferable that ammonia desorption amount in a region of from 100 to 250° C. is 40% or more of the entire ammonia desorption amount (a region of from 25 to 700° C.) and it is more preferable to be 50% or more.

Additionally, the ammonia desorption amount in a region of from 300 to 450° C. to the ammonia desorption amount in a range of from 100 to 300 is 0.6 time or less, preferably 0.5 time or less, more preferably 0.3 time or less, and particularly preferably 0.2 time or less. Above all, it is preferable that the ammonia desorption amount in a region of from 400 to 700° C. is 2 mmol/g or less, preferably 1 mmol/g or less, more preferably 0.5 mmol/g or less, and particularly preferably 0.4 mmol/g or less. It is further preferable even in the case that the ammonia desorption amount in a region of from 100 to 300° C. is 0.1 mmol/g or more, preferably 0.2 mmol/g or more, and more preferably 0.3 mmol/g or more.

(3) Thermogravimetry (TG).

The solid acid catalyst used in the present reaction is a solid acid catalyst that desorbs water in an amount of preferable 3% or more, and more preferably 5% or more, of a reference weight in between 32° C. and 250° C., and further preferably 5% or more of a reference weight in between 50° C. and 200° C., in thermogravimetry (TG). Analytical method and reference weight in TG in this case are according to the methods in the Examples described hereinafter.

The solid acid catalyst used in the present reaction can be selected using measurement of acid strength by Hammett's indicator, temperature programmed desorption of ammonia or thermogravimetry, and a polyether polyol having less coloration can be obtained with high polyether polyol selectivity.

When a part of the acid in the solid acid catalyst satisfying at least one of the above-described requirements is substituted with a metal element, or is modified with a metal element or an organic base, or in addition to the above solid acid catalyst, a component of a metal element-containing compound or an organic base is further added in the form of coexisting in the reaction system, its effect is increased.

The reason that acid strength of the solid acid catalyst, the amount of strong acid point, and desorption behavior of water give influence in the dehydrocondensation reaction of a polyol is not clarified, but it is considered as follows.

Specifically, the solid acid catalyst has sites having various acid properties, and acid properties of the catalyst are not homogeneous. Further, because strong acid is active in the case of the homogeneous catalyst, it was considered in the present reaction that a catalyst having strong acid properties is preferable even in the case of the heterogeneous catalyst. However, it was clarified from the investigations by the present inventors that too strong acid point accelerates a side reaction such as intramolecular dehydration, and therefore was clarified that, for example, ZSM-5 having strong acid properties is unsuitable.

Where a solid acid catalyst in which such a strong acid point is deactivated and that is constituted of an acid point effective for the present reaction is used, a polyether polyol can be obtained with high selectivity. A side reaction is suppressed, and as a result, coloration of the polyether polyol itself is reduced.

Such a solid acid catalyst is the catalyst having an acid function H₀ measured by Hammett's indicator adsorption method of −3 or larger, the catalyst that ammonia desorption amount in a region of from 100 to 350° C. is 60% or more of the entire ammonia desorption amount (a region of from 25 to 700° C.) in the temperature programmed desorption of ammonia, or the solid acid catalyst that desorbs water in an amount of 3% or more of a reference weight in between 30° C. and 250° C. in thermogravimetry, of the present invention.

It is considered that the fact that those solid acid catalysts desorb water in an amount of 3% or more of a reference weight in a temperature range of from 32 to 250° C. has the following meaning.

In the present reaction, water is formed by dehydrocondensation reaction. Further, the reaction temperature is from 120 to 250° C. The fact that water desorbs in a temperature range of from 32 to 250° C. in thermogravimetry (TG) is nothing else that there are release of water and holding of water near the reaction temperature. Contrary to this, the fact that there is no desorption in this temperature region means that water that can participate in reaction is not present around the catalyst. Water has the action to poison acid points of the solid acid catalyst. Therefore, it is considered that if the catalyst holds water in the reaction temperature region, too strong acid points are inactivated, and only good acid points participate in the reaction, thereby the polyether polyol selectivity is improved.

Further, it is considered that substitution and modification with a metal element or a base in those solid acid catalysts, and addition of those components to the reaction system further deactivates minute strong acid points on the solid acid catalyst, and further suppresses side reaction sites.

The solid acid catalyst is not particularly limited so long as it is satisfied with at least one of the above (1) to (3). Preferably, an intercalation compound such as activated clay, zeolite, a mesoporous substance, a metal composite oxide such as silica-alumina or silica zirconia, an oxide or a composite oxide, containing a sulfonic acid group, a carbon material containing a sulfonic acid group, an organic compound such as an ion-exchange resin, a resin having a perfluoroalkylsulfonic acid group at a side chain, and the like can be used. Of those, an intercalation compound such as activated clay, zeolite, a mesoporous substance, a metal composite oxide such as silica-alumina or silica zirconia, a metal oxide or a metal composite oxide, containing a sulfonic acid group, and a carbon material containing a sulfonic acid group are more preferable from the point of being stable under the reaction conditions. Considering the points of having high activity and being inexpensive, an intercalation compound such as activated clay, zeolite, a mesoporous substance, a metal oxide or a metal composite oxide containing a sulfonic acid group, and a carbon material containing a sulfonic acid group are more preferable, and a metal oxide or a metal composite oxide containing a sulfonic acid group, and a carbon material containing a sulfonic acid group are particularly preferable.

When those solid acid catalysts are synthesized, those can be synthesized by the conventional methods.

<Metal Element>

As the metal element that can be substituted with a proton of the acid point of the solid acid catalyst and the metal element that can be modified, alkali metals, alkaline earth metals, transition metals of Group 3 to 12, and elements of Group 13 are preferable, alkali metals and alkaline earth metals are more preferable, and alkali metals are particularly preferable. As the alkali metal, Li, Na, K and Cs are preferable, and Na is particularly preferable.

The content of the metal element is preferably 0.01 equivalent or more, and more preferably 0.05 equivalent or more; and preferably 2.5 equivalents or less, more preferably 1 equivalent or less, and further preferably 0.5 equivalent or less, in terms of metal element, to the acid amount of the solid acid catalyst.

The term “acid amount” used herein means a theoretical acid amount or an acid amount obtained by a neutral salt decomposition method. In the case of zeolite comprising Al and silicon, the acid amount means a theoretical acid amount calculated from the amount of Al, and in the case of zeolite containing elements other than Al, a mesoporous substance, a sulfonic acid-containing solid catalyst, an oxide, a composite oxide, and an intercalation compound such as activated clay, the acid amount means an acid amount obtained by a neutral salt decomposition method.

The “neutral salt decomposition method” used herein means that a solid acid catalyst is ion-exchanged with a saturated sodium chloride aqueous solution at 20 to 25° C. for 15 minutes, and H⁺ ion-exchanged with Na⁺ is determined by titrating with a sodium hydroxide aqueous solution having a known concentration.

In the case of a solid acid catalyst, there is the case to previously obtain a catalyst having an acid point substituted with a metal. The content of the metal element means an equivalent to the original acid amount in the case that the metal element is not substituted with an acid point of a solid acid.

Specifically, when the acid amount in the case of metal unsubstitution is 1 mmol/g and the acid amount in the case of metal substitution is 0.7 mmol/g, the content of metal element of a metal-substituted solid acid is 0.3 equivalent to the acid amount. Further, the substitution amount of a metal can be obtained by an elementary analysis.

Metal element source can use a metal element-containing compound, and specific examples thereof include salts of mineral acids such as sulfuric acid salts, hydrogen sulfates, nitric acid salts, halides, phosphoric acid salts, hydrogen phosphates and boric acid salts of metals; organic sulfonic acid salts such as trifluoromethanesulfonic acid salts, p-toluenesulfonic acid salts and methanesulfonic acid salts; metal salts of carboxylic acid salts or the like such as formic acid salts and acetic acid salts; metal hydroxides, metal alkoxides, and acetyl acetonates of metals.

A method of substitution or modification of a metal element to the solid acid catalyst can use the conventional methods such as a method of ion-exchanging a solid acid catalyst in an solution of the desired metal compound, a method of impregnating and forcedly carrying, and a method of pore-filling a solution of a metal compound and drying. If necessary, those catalysts thus obtained can be applied to a treatment such as water washing, drying and baking.

<Organic Base>

The organic base is preferably a nitrogen-containing organic base, particularly a nitrogen-containing organic base having tertiary or quaternary nitrogen atom. Examples of the base include nitrogen-containing heterocyclic compounds having a pyridine skeleton, such as pyridine, picoline, quinoline and 2,6-lutidine; nitrogen-containing heterocyclic compounds having N—C═N bond, such as N-methylimidazole, 1,5-diazebicyclo[4.3.0]-5-nonene and 1,8-diazabicyclo-[5.4.0]-7-undecene; trialkylamines such as triethylamine and tributylamine; and quaternary ammonium salts such as 1-methylpyridium chloride. Of those, the compounds having a pyridine skeleton and compounds having N—C═N bond are preferable, and the compounds having a pyridine skeleton are particularly preferable.

The organic base is used in an amount of preferably 0.01 equivalent or more, and more preferably 0.05 equivalent or more; and preferably 2.5 equivalents or less, more preferably 1 equivalent or less, and particularly preferably less than 1 equivalent, to the acid amount of the solid acid catalyst (in this case, indicating the acid amount in the case of unsubstitution as same as the case of the metal element).

A method of modifying a metal element to the solid acid catalyst can use the conventional methods such as a method of mixing a solid acid catalyst in an solution containing the desired base, a method of impregnating and forcedly carrying, and a method of pore-filling a base-containing solution and drying. If necessary, those catalysts thus obtained can be applied to a treatment such as water washing, drying and baking.

<Combined Use of Solid Acid Catalyst and Metal Element and/or Organic Base>

The present invention may further use a metal element and/or an organic base in addition to the solid acid catalyst satisfying at least one of the requirements of (1) a catalyst having the acid function H₀ measured by Hammett's indicator adsorption method of larger than −3, (2) a catalyst having the desorption amount of ammonia in a region of from 100 to 350° C. of 60% or more of the entire ammonia desorption amount (a region of from 25 to 700° C.) in temperature programmed desorption measurement of ammonia, and (3) a catalyst that water desorbs in an amount of 3% by weight or more of a reference weight in between 32 and 250° C. in thermogravimetry. Specifically, those may be added to the reaction system separately, or those compounds may be mixed and then used in the reaction. In this case, the total amount of the entire metal elements and the organic bases, present in the reaction system is made to be fallen within the above range.

The solid acid catalyst and the metal element or the organic base may be present in the reaction system separately, or the solid acid catalyst and the metal element or the organic base may form a salt.

The metal element and the organic base that can be used in this case can be the same materials as described above.

<Raw Material Polyol>

The polyol as the reaction raw material preferably uses a diol having two primary hydroxyl groups, such as 1,3-propanediol, 2-methyl-1,3-propanediol, 2,2-dimethyl-1,3-propanediol, 1,6-hexanediol, 1,7-heptanediol, 1,8-octanediol, 1,9-nonanediol, 1,10-decanediol or 1,4-cyclohexanedimethanol, and more preferably uses 1,3-propanediol. However, even though the diol having two primary hydroxyl groups, ethylene glycol, 1,4-butanediol, 1,5-pentanediol and the like form a cyclic ether ether by dehydrocondensation reaction as described before, and therefore are not preferable as the raw material in the method of the present invention. Generally, those diols are used alone, but if desired, can be used as a mixture of two or more thereof. Even in this case, however, it is preferable that the main diol occupies 50 mol % or more. Further, an oligomer of from a dimer to a nonamer obtained by dehydrocondensation of the main diol can be used in combination with those diols. Moreover, a polyol of a triol or more, such as trimethylolethane, trimethylolpropane and pentaerythritol, or an oligomer of those polyols can be used together. Even in this case, however, it is preferable that the main diol occupies 50 mol % or more. In general, diols that form a 5-membered or 6-membered cyclic ether by dehydrocondensation reaction, such as 1,4-butanediol and 1,5-pentanediol, are excluded, and diols of from 3 to 10 carbon atoms having two primary hydroxyl groups, or a mixture of the diol and other polyol, wherein the proportion of the other polyol is less than 50 mol %, is used in the reaction. Preferably, a diol selected from the group consisting of 1,3-propanediol, 2-methyl-1,3-propanediol and 2,2-dimethyl-1,3-propanediol, or a mixture of the diol and other polyol, wherein the proportion of the other polyol is less than 50 mol %, and particularly preferably, 1,3-propanediol, or a mixture of this and other polyol, wherein the proportion of the other polyol is less than 50 mol %, is used in the reaction.

<Production Method of Polyether Polyol>

In the present invention, the production of a polyether polyol by dehydrocondensation of a polyol can be conducted in a batchwise system or a continuous system. In the case of a batchwise system, a polyol as a raw material, a solid acid catalyst and if necessary, a metal element or an organic base are charged in a reactor, and reacted under stirring. In this case, the solid acid catalyst is used in an amount of generally form 0.01 to 1 time by weight to the polyol as a raw material.

In the case of a continuous reaction, the following method can be used. For example, a solid acid catalyst is retained in a reaction apparatus having many stirring tanks in series or in a flow type reaction apparatus, a polyol as a raw material is continuously supplied to the reactor, and only a reaction liquid that does not contain the solid acid catalyst is taken out of other end. In this case, a suspended bed and/or a fixed bed reaction can be employed.

In general, the raw material polyol is supplied for 1 hour in an amount that the lower limit is generally 0.01 time by weight or more, and preferably 0.1 time by weight or more, and the upper limit is generally 10,000 times by weight or less, and preferably 1,000 times by weight or less, to the solid acid catalyst retained in the reaction apparatus. In this case, there is the case that equivalent ratio of the base to the solid acid catalyst in the reaction apparatus decreases with the lapse of time. Therefore, according to need, the solid acid catalyst is taken out with small portions, and a fresh catalyst is charged, or a base is supplied together with the raw material polyol so that the equivalent ratio of the organic base to the acid maintains at the desired value.

The temperature of dehydrocondensation is that the reaction is conducted at a temperature such that the lower limit is generally 120° C. or higher, and preferably 140° C. or higher, and the upper limit is generally 250° C. or lower, and preferably 200° C. or lower. The reaction is preferably conducted under an inert gas atmosphere such as nitrogen or argon. Reaction pressure is optional so long as it is in a range such that the reaction system maintains a liquid phase, and the reaction is generally conducted under atmospheric pressure. If desired, the reaction may be conducted under reduced pressure, or an inert gas may be flown through the reaction system, in order to accelerate removal of water produced by the reaction from the reaction system.

The reaction time varies depending on the amount of catalyst used, the reaction temperature, and the desired yield or properties of the dehydrocondensated product, but the lower limit thereof is generally 0.5 hour or more, and preferably 1 hour or more, and upper limit thereof is generally 50 hours or less, and preferably 20 hours or less. The reaction is generally conducted in non-solvent, but if desired, a solvent can be used. The solvent is used by appropriately selecting from organic solvents used in the conventional organic synthesis reaction, considering vapor pressure, stability, solubility of raw material and product, under the reaction conditions.

Separation and recovery of the produced polyether polyol from the reaction system can be conducted in the conventional manners.

In the case of the suspended bed reactor, the suspended solid acid catalyst is removed from the reaction liquid by filtration or centrifuge. In the case of adding a metal compound to the reaction system, a method of removing by water washing, or a method of forming a sparingly soluble salt; and removing by filtration can be employed. In the case of the organic base, where distillation is possible, it is removed by distillation operation, and the organic base taken out can be returned to the reaction system. Where distillation is impossible, it can be removed by water washing.

According to need, a low boiling oligomer is removed by distillation or extraction with water or the like, thereby obtaining the desired polyether polyol.

In the case of the fixed bed reaction, a low boiling component or a low boiling oligomer is removed from the reaction liquid taken out, by distillation or water washing according to need, thereby obtaining the desired polyether polyol.

If necessary, those polyether polyols further undergo a drying step, thereby obtaining products.

<Polyether Polyol>

The polyol polyether obtained by the method of the present invention is preferably with less coloration. The degree of coloration is visually the order of black>brown>yellow>1colorless (white).

Number average molecular weight of the polyether-polyol of the present invention can be controlled by the kind of catalyst used, and the amount of catalyst. The lower limit is generally 80 or more, preferably 600 or more, and more preferably 1,000 or more, and the upper limit is generally 10,000 or less, preferably 7,000 or less, and more preferably 5,000 or less.

The molecular weight distribution (weight average molecular weight/number average molecular weight) is preferable as approaching 1, and the upper limit is generally 3 or less, and preferably 2.5 or less.

The polyether polyol of the present invention can be used to applications of elastic fibers, thermoplastic polyester elastomers, thermoplastic polyurethane elastomers, coating materials and the like.

### EXAMPLE 1

The present invention is described in more specifically below by the Examples.

<Measurement Method of Acid Amount by Neutral Salt Decomposition Method>

Measurement of an acid amount by a neutral salt decomposition method was conducted as follows. 10 mg of a sample was precisely weighed to one place of decimal, and 30 ml of a saturated sodium chloride aqueous solution (prepared by special grade sodium chloride, a product of Junsei Chemical Co., and desalted water) was added, followed by stirring with a stirring bar at room temperature for 15 minutes.

Thereafter, a solid acid catalyst was filtered off, and washed with desalted water. The filtrate was titrated with 0.025M sodium hydroxide aqueous solution to determine the amount of proton ion-exchanged, and an acid amount per unit weight was determined.

<Acid Strength Function Measurement Method of Hammett>

0.1 g of a solid acid catalyst was held at room temperature (25° C.±3° C.) under saturated water vapor to adsorb the saturated water vapor. 2 ml of special grade benzene, a product of Kokusan Chemical Co., was added to the catalyst, and two droplets of Hammett's indicators in 0.1 wt % solution were added dropwise thereto. Change in color on the catalyst was observed.

The Hammett's indicators were added dropwise in the order of anthraquinone (pKa−8.2)→benzalacetophenone (Pka−5.6)→+dicinnamalacetone (pKa−3.0)→>4-benzeneazodiphenylamine (pKa +1.5)→p-dimethylaminoazobenzene (pKa+3.3)→4-benzeneazo-1-naphthylamine (pKa+4.0)→methyl red (pKa+4.8)→neutral red (pKa+6.8).

The term “acid function H₀ measured by Hammett's indicator adsorption method is larger than −3” means, for example, that there is no coloration into acidic color up to anthraquinone (pKa−8.2), benzalacetophenone (Pka−5.6) and dicinnamalacetone (pKa−3.0), and there is coloration into acidic color by the indicators of 4-benzeneazodiphenylamine (pKa+1.5), p-dimethylaminoazobenzene (pKa+3.3), 4-benzeneazo-1-naphthylamine (pKa+4.0), methyl red (pKa +4.8) and neutral red (pKa+6.8), having pKa larger than −3.0, or there is no coloration into acidic color by any indicators.

Inversely, the term “acid function H₀ is smaller than −3” means, for example, that there is coloration into acidic color by an indicator having pKa smaller than dicinnamalacetone (pKa−3.0), such as anthraquinone (pKa−8.2) or benzalacetophenone (Pka−5.6).

In Table 1, for example, in the case that there is no coloration into acidic color by 4-benzeneazodiphenylamine (pKa+1.5), and there is coloration by p-dimethylaminoazobenzene (pKa+3.3), it is expressed “+1.5<H₀<3.3”. In the case that there is no coloration into acidic color even by pKa+6.8, the acid strength is expressed H₀>+6.8. H₀ of each catalyst is shown in Table 1.

<Temperature Programmed Desorption (TPD) of Ammonia>

Temperature programmed desorption of ammonia was conducted by the following method.

Measurement device: Anelva Co., AGS-7000, EI Method, 70 eV

Method: TPD-MS (Temperature Programmed Desorption

Mass-Spectrometry)

Measurement condition:

Sample amount; precisely weighing about 30 mg

Pretreatment condition of sample; He 80 ml/min, rising from room temperature to 250° C. at 30° C./min, and then holding at 250° C.×30 minutes.

Ammonia adsorption condition; “after vacuum evacuating a sample with a rotary pump at 100° C., injecting ammonia gas (purity 100%) of 90 torr portion in the sample at the same temperature, and holding for 15 minutes”→“vacuum evacuation 100° C.×30 minutes”→“He 200 ml/min, 100° C.×30 minutes”→“5 minutes later after returning to room temperature, starting TDS measurement”

TPD measurement gas; He 80 ml/min

TPD measurement temperature range; from room temperature to 700° C. (10° C./min temperature rising)

Quantitative determination of desorbed ammonia amount; separately from the measurement of each sample, a constant amount of ammonia was injected, and calibration curve was prepared from its amount (mole number) and an area of ionic strength (m/z=16) at that time. The desorbed ammonia amount of each sample was calculated and quantitatively determined from the previously determined calibration curve by obtaining the area of ionic strength at the corresponding temperature range.

In the case that a sample has the possibility of having m/z=16 originated from other compound (representative example: water) in an amount that cannot disregard to m/z=16 originated from ammonia, ionic strength of m/z=16 originated from such a compound is calculated, and it is necessary to remove its contributory extent from the ammonia quantitative value.

The ionic strength of m/z=16 of the compound can calculate ionic strength of m/z=16 originated from the compound in TPD measurement data from the ratio of the original “ionic strength of m/z other than m/z=16” and “ionic strength of m/z=16” of the compound.

<Thermogravimetry (TG)>

The thermogravimetry was conducted by the following method.

Sample; after pretreatment of holding in saturated water vapor for 2 days, sampling in air at room temperature.

Measurement device; SII Nano Technology Inc., TG-DTA 6300

Calibration of temperature; correcting with three kinds of metals of In, Pb and Sn

Calibration of weight; conducting calibration with a weight at room temperature. Conducting calibration with calcium oxalate.

Amount of sample; about 10 mg

Sample vessel; made of Al, 5 mm diameter×2.5 mm

Measurement method; flowing dried nitrogen gas (purity 99.999% or more, dew point −60° C.) at 200 ml/min, holding at room temperature of 30° C. for 30 minutes, and rising temperature to 500° C. at a rate of 10° C./min.

Reference weight; weight obtained by subtracting weight loss up to 32° C. from the weighed weight of a sample.

Desorption amount of water; proportion (%) of weight loss in a predetermined temperature range to reference weight.

The results of thermogravimetry (TG) are shown in Table 1.

<Elemental Analysis>

With respect to the amount of Al, Na and Si contained in a zeolite catalyst, numerical values quantitatively determined by the following method were employed, unless otherwise indicated.

XRF method: A sample was dried at 120° C. for 2 hours. After standing the sample to cool, 500 mg was batched off, and mixed with 5.00 g of LiB₄O₇. The resulting mixture was melted, cooled and molded into glass beads, followed by quantitative determination with fluorescent X ray (XRF (fundamental parameter method: FP method)).

Because ZSM-5 (silicalite) used in Example 4 had small Al and Na content, quantitative determination was conducted by the following method.

Chemical analysis method: A sample was dried at 120° C. for 2 hours. After standing the sample to cool, apiece thereof was batched off. The entire amount was decomposed with a wet decomposition method, and its solution was prepared, followed by quantitative determination with ICP-AES (Al; calibration curve method) and AAS (Na, calibration curve method).

The amount of Na and K in sulfonic acid-containing silica and Nafion was analyzed by the following method.

Chemical analysis method (2): A sample was dried at 120° C. for 2 hours. After standing the sample to cool, apiece thereof was batched off. The entire amount was decomposed with a dry ash method, and its solution was prepared, followed by quantitative determination with AAS (calibration curve method).

<Measurement of Number Average Molecular Weight (Mn)>

Measurement of a number average molecular weight of a polyether polyol was conducted by gel permeation chromatography under the following conditions, and calculation was made on the basis of a polytetrahydrofuran.

TSK-GEL GMHXL-N (7.8 mm ID×30.0 cm L) (Tosoh Corporation)

Correction of mass:

POLYTETRAHYDROFURAN CALIBRATION KIT (Polymer Laboratories)

(Mp=547000, 283000, 99900, 67500, 35500, 15000, 6000, 2170, 1600, 1300)

Solvent: Tetrahydrofuran

<GC Analysis Condition>

1,3-Propanediol contained in a light boiling component and an oily layer obtained was analyzed with gas chromatography (GC).

Column: HR-20M film thickness 0.25 μm, 0.25 mmID×30 m

Carrier: nitrogen about 1.5 ml/min, split ratio about 40

Oven temperature: 50° C.−(10° C./min temperature rising)−230° C. (holding for 10 minutes)

Inlet, detector temperature: 240° C.

Internal standard: n-tetradecane

In the following Examples, catalysts used in Examples 1, 2 and 8 and Comparative Examples 1 and 3 were previously dried at 300° C. for 12 hours, and then used.

### EXAMPLE 1

### Distillation Purification of 1,3-propanediol

250 g of 1,3-propanediol (reagent manufactured by Aldrich, purity 98%, Batch #10508AB) and 1.75 g of potassium hydroxide were placed in a 500 ml four-necked flask made of Pyrex (registered trade mark) equipped with a reflux condenser, a nitrogen introduction pipe and a thermometer under nitrogen atmosphere. The flask was heated in an oil bath, and when liquid temperature reached 162° C., the temperature was maintained at 162 to 168° C. 2 hours later, the flask was taken out of the oil bath, and allowed to stand to cool to room temperature. Simple distillation was conducted at about 90° C. under reduced pressure. 11 g of a forerun was discarded, and about 230 g of a distillate was recovered.

<Dehydrocondensation Reaction of 1,3-propanediol>

20 g of 1,3-propanediol distilled and purified by the above method was placed in a 100 ml four-necked flask made of Pyrex (registered trade mark) equipped with a distillation pipe, a nitrogen introduction pipe, a thermometer and a mechanical stirrer while supplying nitrogen at 40 Nml/min. This flask was dipped in an oil bath at 25° C., and 10 g of HY Zeolite (HSZ-320HOA, SiO₂/Al₂O₃ (molar ratio)=5.3, lot. 2001) manufactured by Tosoh Corporation was gradually added while stirring.

After adding a solid acid catalyst, the resulting mixture was stirred at room temperature for 10 minutes. After sufficiently removing oxygen in a reactor, temperature of the oil bath was set to 200° C., and heating was initiated. Temperature of the reactor was controlled to 185° C.±3° C. and maintained for 6 hours to conduct reaction. The flask was taken out of the oil bath, and was allowed to stand to cool to room-temperature. Water formed during reaction was flown out by accompanying with nitrogen. The reaction liquid was collected together with a by-product and 1,3-propanediol using a trap whose circumference was cooled with a dry ice-ethanol solution. Those were considered a light boiling component, and the amount of 1,3-propanediol contained was separately analyzed by gas chromatography.

50 g of tetrahydrofuran was added to the reaction liquid cooled to room temperature, and after stirring for 1 hour, the solid acid catalyst was filtered off with a PTFE-made filter of 1 μm. Inside of the reactor and inside of the filter were washed using 30 ml of tetrahydrofuran (containing 0.03 wt % BHT (butylhydroxytoluene)), and this washing solution was mixed with the above filtrate. This operation was repeated two times, and tetrahydrofuran was distilled off from the collected organic layer under reduced pressure. The oil layer obtained was heated to 50° C., and dried under vacuum of 2 to 3 mmHg for 3 hours. This oil layer was measured with gel permeation chromatography to determine a number average molecular weight (Mn). Further, the amount of 1,3-propanedoil contained in this oil layer was analyzed with gas chromatography and quantitatively determined.

The amount of unreacted 1,3-propanediol was determined in the light-boiling component and the component in oil layer, respectively, and the total amount was obtained. Conversion of 1,3-propanediol was determined by the following equation. Selectivity of a polyether polyol was determined by the following equation in a manner such that the amount of 1,3-propanediol is subtracted from the oil layer obtained, and the remainder is considered the polyether polyol.

<Conversion of Raw Material 1,3-protanediol>
(Conversion of 1,3-propanediol)(%)=
{(mole number of 1,3-propanediol charged)−(mole number
of residual 1,3-propanediol)}×100/(mole number of
1,3-propanediol charged)<
Selectivity of Polyether Polyol>
(Selectivity of polyether polyol)(%)=
[{(weight(g)of oil layer/Mn)×(Mn−18)/58}−(mole number
of 1,3-propanediol in oil layer)]×100/(mole number of
1,3-propanediol converted)

The results are shown in Table 1.

### EXAMPLE 2

A polytrimethylene ether glycol was obtained in the same manner as in Example 1, except for using USY Zeolite manufactured by Tosoh Corporation (HSZ-330HUA, Na₂O/SiO₂/Al₂O₃ (molar ratio)=0.02/6/1 (nominal value by manufacturer) lot. C2-0719) as the solid acid catalyst. The results are shown in Table 1.

### EXAMPLE 3

### Preparation Method of Metal Element-Substituted Solid Acid

11 g of sodium nitrate, special grade, manufactured by Kishida Chemical Co., was placed in a four-necked flask made of Pyrex (registered trade mark) equipped with a mechanical stirrer. 100 g of desalted water was added and dissolved while stirring. Thus, about 100 ml of 1.3 mol/liter sodium nitrate aqueous solution was prepared. Further, while stirring, 20 g of the same USY Zeolite (HSZ-330HUA) manufactured by Tosoh Corporation as used in Example 2 was added, and the liquid temperature was maintained at 80° C. for 2 hours. Zeolite was filtered off, and washed with desalted water of 80° C. After air drying and then drying in a drier at 120° C. for 12 hours, the zeolite was baked in air at 500° C. for 2 hours to obtain Na partially exchanged USY zeolite. As a result of elemental analysis, it was found to be Na₂O/SiO₂/Al₂O₃ (molar ratio)=0.07/6.4/1.

<Dehydrocondensation Reaction of 1,3-propanediol>

A polytrimethylene ether glycol was obtained in the same manner as in Example 1, except for using the above catalyst as the solid acid catalyst. The results are shown in Table 1.

### EXAMPLE 4

### Preparation Method of Metal Element-Substituted Solid Acid

7.8 g of ammonium nitrate, special grade, manufactured by Kishida Chemical Co., was placed in a four-necked flask made of Pyrex (registered trade mark) equipped with a mechanical stirrer. 100 g of desalted water was added and dissolved while stirring. Thus, about 100 ml of 0.95 mol/liter ammonium nitrate aqueous solution was prepared. Further, while stirring, 13 g of ZSM-5 (silicalite) manufactured by N.E. Chemcat Corporation (K-MCM-04, Na₂O/SiO₂/Al₂O₃ (molar ratio)=21/1640/1 (analytical value by manufacturer)) was added, and the liquid temperature was maintained at 80° C. for 2 hours. Zeolite was filtered off, and washed with desalted water of 80° C. After air drying and then drying in a drier at 120° C. for 12 hours, the zeolite was baked in air at 500° C. for 2 hours to obtain Na partially exchanged silicalite. As a result of elemental analysis, it was found to be Na₂O/SiO₂/Al₂O₃ (molar ratio)=0.14/1446/1.

<Dehydrocondensation Reaction of 1,3-propanediol>

A polytrimethylene ether glycol was obtained in the same manner as in Example 1, except for using the above catalyst as the solid acid catalyst. The results are shown in Table 1.

### COMPARATIVE EXAMPLE 1

A polytrimethylene ether glycol was obtained in the same manner as in Example 1, except for using 40.5 g of 1,3-propanediol, using 16.6 g of ZSM-5 zeolite manufactured by N.E. Chemcat Corporation (K-MCM-02-2, Na₂O/SiO₂/Al₂O₃ (molar ratio)=0/47/1) as the solid acid catalyst, and supplying nitrogen at 100 Nml/min. The results are shown in Table 1.

As a result of measurement of TPD of this catalyst, the ammonia desorption amount at 100 to 250° C. was 0.19 mmol/g, and this was 33% of the overall ammonia desorption amount (a region of from 25 to 700° C.). Further, the ammonia desorption amount at 100 to 300° C. was 0.25 mmol/g, and this was 43% of the overall ammonia desorption amount (a region of from 25 to 700° C.). The ammonia desorption amount at 100 to 350° C. was 0.33 mmol/g, and this was 57% of the overall ammonia desorption amount (a region of from 25 to 700° C.).

Further, the ammonia desorption amount at 300 to 450° C. was 0.24 mmol/g. At that time, the ammonia desorption amount in a region of from 300 to 450° C. to the ammonia desorption amount in a region of from 100 to 300° C. was 0.96 time. The desorption amount of ammonia desorbed in a region of from 400 to 700° C. was 0.15 mmol/g.

<Preparation Method of Metal Element-Substituted Solid Acid>

Na partially exchanged ZSM-5 zeolite was obtained in the same manner as in the preparation method of metal element-substituted solid acid in Example 3, except for using 25 g of sodium nitrate, 280 g of desalted water, and 30 g of ZSM-5 zeolite used in Comparative Example 1 as the solid acid catalyst.

As a result of elemental analysis, it was found to be Na₂O/SiO₂/Al₂O₃ (molar ratio)=0.26/49/1)

<Dehydrocondensation Reaction of 1,3-propanediol>

A polytrimethylene ether glycol was obtained in the same manner as in Example 1, except for using the above catalyst as the solid acid catalyst. The results are shown in Table 1.

### EXAMPLE 5

### Preparation Method of Metal Element-Substituted Solid Acid

10 g of desalted water was added to 4 g of Nafion NR-50 (Beads 7-9 mesh), a reagent manufactured by Aldrich, and 3.3 ml of 1N—NaOH aqueous solution was added dropwise using measuring pipette. After stirring for 2 hours, the resulting mixture was washed with 100 ml of desalted water, dried and then dried at 50° C. under reduced pressure of 2 mmHg. Acid amount by neutral salt decomposition method was 0.11 mmol/g. Because the acid amount of Nafion used in Example 5 was 0.9 mmol/g, 88% of H⁺ was substituted with Na⁺.

<Dehydrocondensation Reaction of 1,3-propanediol>

A polytrimethylene ether glycol was obtained in the same manner as in Example 1, except for using 2.5 g of the above catalyst as the solid acid catalyst, heating the oil bath to 182° C., controlling the reaction temperature to 169° C.±3° C., and conducting separation of the catalyst by decantation.

The results are shown in Table 1.

### EXAMPLE 6

### Preparation Method of Solid Acid

20 g of mercaptopropyltrimethoxysilane oligomer (X-41-1805, lot 305006) manufactured by Shin-Etsu Chemical Co., Ltd., and 39 g of ethanol, special grade, manufactured by Junsei Chemical Co. were added to a 100 ml three-necked flask made of Pyrex (registered trade mark) equipped with mechanical stirrer, and while stirring, 1.7 g of desalted water was added, followed by stirring at room temperature for 30 minutes. Thereafter, while stirring, temperature in the flask was maintained at 70° C. for 20 hours to proceed hydrolysis, and gelation gradually proceeded. After once returning to room temperature, the product was taken out and placed in a 100 ml egg-plant flask. Solvent was distilled off, and after drying, the product was ground. After forming a powder with a mortar, the powder was dried at 70° C. under 2 mmHg for 3 hours.

13 g of the powder was placed in a 100 mol three-necked flask, and while stirring with a mechanical stirrer, 36 g of 30% hydrogen peroxide solution was added dropwise over 4 hours. During the dropwise addition, heat generation occurred. Therefore, while cooling with a water bath, SH group was oxidized into SO₃H group.

After allowing to stand at room temperature for 12 hours, the reaction mixture was stirred at 70° C. for 4 hours to age. After cooling to room temperature, 1M sulfuric acid aqueous solution was introduced such that concentration of the solid acid is 1 wt % to conduct ion-exchange. The resulting mixture was washed with water, dried and dried under reduced pressure of 16 mmHg for 3 hours to obtain sulfonic acid group-containing silica. The acid amount by neutral salt decomposition method was 1.3 mmol/g. By elemental analysis, it was found that the sum of the substitution amount of Na⁺ and K⁺ is 0.002 equivalent to the acid amount. As a result of measurement of TPD of this catalyst, the ammonia desorption at 100 to 250° C. was 0.83 mmol/g, and this was 53% of the overall ammonia desorption amount (a region of from 25 to 700° C.). The ammonia desorption at 100 to 300° C. was 1.1 mmol/g, and this was 69% of the overall ammonia desorption amount (a region of from 25 to 700° C.). The ammonia desorption at 100 to 350° C. was 1.2 mmol/g, and this was 76% of the overall ammonia desorption amount (a region of from 25 to 700° C.).

Further, the ammonia desorption amount at 300 to 450° C. was 0.15 mmol/g. At that time, the ammonia desorption amount in a region of from 300 to 450° C. to the ammonia desorption amount in a region of from 100 to 300° C. was 0.14 time.

The desorption amount of NH₃ desorbed in a region of from 400 to 700° C. was 0.34 mmol/g.

<Hydrocondensation Reaction of 1,3-butanediol>

A polytrimethylene ether glycol was obtained in the same manner as in Example 1, except for using 5 g of the above catalyst as the solid acid catalyst, and controlling the reaction temperature to 189° C.±3° C. The results are shown in Table 1.

### EXAMPLE 7

### Preparation Method of Metal Element-Substituted Solid Acid

10 g of desalted water was added to 6 g of the catalyst used in Example 7, and while stirring at room temperature, 0.66 ml of 1N—NaOH was added dropwise at room temperature, followed by stirring for 2 hours. The catalyst was filtered off, and washed with 100 ml of desalted water. This operation was repeated two times, and the catalyst was similarly washed with desalted water, dried, and dried at room temperature under reduced pressure of 16 mmHg to obtain Na-substituted sulfonic acid group-containing silica. The acid amount by neutral salt decomposition method was 0.70 mmol/g. Therefore, the substitution amount of Na⁺ is 0.45 equivalent to the original acid amount.

<Dehydrocondensation Reaction of 1,3-propanediol>

A polytrimethylene glycol was obtained in the same manner as in Example 6, except that 20 g of 1,3-propanediol was placed in a four-necked flask, and 0.0514 g (0.65 mmol) of pyridine, special grade, manufactured by Junsei Chemical Co., was added, after sufficiently stirring, the flask was dipped in a 25° C. oil bath, and while stirring, 5.06 g of the above catalyst was added as the solid acid catalyst. The sum of Na⁺ and pyridine is 0.55 equivalent to the original acid amount. The results are shown in Table 1.

### EXAMPLE 8

A polytrimethylene glycol was obtained in the same manner as in Example 7, except that 0.34 g (0.14 time equivalent to the theoretical acid amount) of pyridine, and 10 g of USY Zeolite manufactured by Tosoh Corporation (HSZ-350HUA, Na₂O/SiO₂/Al₂O₃ (molar ratio)=0.01/9.2/1, lot. C2-1X05) as the solid acid catalyst were gradually added, and the reaction temperature was 185±3° C. The results are shown in Table 1.

### COMPARATIVE EXAMPLE 3

A polytrimethylene glycol was obtained in the same manner as in Example 8, except for using 0.26 g (0.5 time equivalent to the theoretical acid amount) of pyridine, and 10 g of the same ZSM-5 Zeolite as used in Comparative Example 1 as the solid acid catalyst. The results are shown in Table 1.

### EXAMPLE 9

A polytrimethylene glycol was obtained in the same manner as in Example 7, except for using 0.18 g (1 time equivalent to acid amount by neutral salt decomposition method) of pyridine, and 2.5 g of the same Nafion NR50, a reagent manufactured by Aldrich, as used in <Preparation method of metal element-substituted solid acid catalyst> in Example 5 as the solid acid catalyst, heating the oil bath to a temperature of 182° C., controlling the reaction temperature to 169° C.±3° C., and conducting separation of the catalyst by decantation. The results are shown in Table 1.

### EXAMPLE 10

A polytrimethylene glycol was obtained in the same manner as in Example 6, except for using 40 g of 1,3-propanediol and 13 g of Nafion Powder (a product of Dupont, XR-500 Powder, 13S49-8055-K⁺ 1200EW, acid amount by neutral salt decomposition method is 0.04 mmol/g. It was found by elemental analysis that the sum of substitution amount of Na⁺ and K⁺ is 0.95 equivalent to the original acid amount.), and supplying nitrogen at 100 Nml/min. The results are shown in Table 1.

### EXAMPLE 11

A polytrimethylene glycol was obtained in the same manner as in Example 7, except for using 0.036 g (0.46 mmol) of pyridine and 5 g of the same Nafion powder used in Example 7 as the solid acid catalyst. The sum of the metal element and the base is 1.1 times equivalent to the original acid amount. The results are shown in Table 1.

### EXAMPLE 12

### Preparation Method of Metal Element-Substituted Solid Acid

48.8 g of ammonium nitrate, special grade, manufactured by Kishida Chemical Co., was placed in a four-necked flask made of Pyrex equipped with a mechanical stirrer. 600 ml of desalted water was added and dissolved while stirring. Thus, about 600 ml of 1 mol/liter ammonium nitrate aqueous solution was prepared. Further, while stirring, 30.3 g of ferrierite manufactured by Tosoh Corporation (HSZ-720KOA (K₂O/Na₂O/SiO₂/Al₂O₃ (molar ratio)=0.23/0.70/17.7/13 (nominal value), lot. 5001) was added, and the liquid temperature was maintained at 80° C. for 2 hours. Zeolite was filtered off, and washed with desalted water of 80° C. This operation was repeated two times. After air drying and then drying in a drier at 120° C. for 12 hours, the zeolite was baked in air at 500° C. for 2 hours to obtain H⁺ type ferrierite.

<Dehydrocondensation Reaction of 1,3-propanediol>

A polytrimethylene ether glycol was obtained in the same manner as in Example 1, except for using the above catalyst as the solid acid catalyst. The results are shown in Table 1.

### COMPARATIVE EXAMPLE 4

### Preparation Method of Metal Element-Substituted Solid Acid

Na partially exchanged ZSM-5 zeolite was obtained in the same manner as in the preparation method of metal element-substituted solid acid in Example 3, except for using 8.5 g of sodium nitrate, 100 g of desalted water, 1 mol/liter sodium nitrate aqueous solution, and 20 g of ZSM-5 zeolite used in Comparative Example 1 as the solid acid catalyst.

As a result of elemental analysis, it was found to be Na₂O/SiO₂/Al₂O₃ (molar ratio)=0.23/51/1.

<Dehydrocondensation Reaction of 1,3-propanediol>

A polytrimethylene ether glycol was obtained in the same manner as in Example 1, except for using the above catalyst as the solid acid catalyst. The results are shown in Table 1.

### EXAMPLE 13

### Preparation Method of Metal Element-Substituted Solid Acid

Na-substituted USY zeolite was obtained in the same manner as in Example 3, except for using 5.36 g of sodium nitrate, 50 ml of desalted water, 1.3 mol/liter sodium nitrate aqueous solution, and 15 g of USY zeolite manufactured by Tosoh Corporation (HSZ-350HUA lot. C2-1X05, Na₂O/SiO₂/Al₂O₃ (molar ratio)=0.01/9.2/1). (Na₂O/SiO₂/Al₂O₃ (molar ratio)=0.12/10/1).

<Dehydrocondensation Reaction of 1,3-propanediol>

A polytrimethylene ether glycol was obtained in the same manner as in Example 1, except for using the above catalyst as the solid acid catalyst. The results are shown in Table 1.

TABLE 1

| | | | | | 1,3-PD conversion |
|---|---|---|---|---|---|
| | Catalyst | [Na + K]/H⁺* | Pyridine/H⁺* | Mn | (%) |
| Example 1 | HY (5.3) | 0.24 | 0 | 113 | 61 |
| Example 2 | USY (6) | 0.02 | 0 | 222 | 98 |
| Example 3 | Na-substituted USY (6.4) | 0.07 | 0 | 146 | 81 |
| Example 4 | ZSM-5 (1446) | 0.14 | 0 | 184 | 90 |
| Comparative | ZSM-5 (47) | 0 | 0 | 314 | 86 |
| Example 1 | | | | | |
| Comparative | Na-substituted ZSM-5 (49) | 0.26 | 0 | 314 | 89 |
| Example 2 | | | | | |
| Example 5 | Na-substituted Nafion NR50 | 0.88 | 0 | 143 | 81 |
| Example 6 | Sulfonic acid group-containing | 0.002 | 0 | 365 | 95 |
| | silica | | | | |
| Example 7 | Na-susbtitututed suflonic | 0.45 | 0.1 | 109 | 66 |
| | acid-containing silica | | | | |
| Example 8 | USY (9.2) | 0.01 | 0.14 | 107 | 58 |
| Comparative | ZSM-5 (47) | 0 | 0.5 | 287 | 99 |
| Example 3 | | | | | |
| Example 9 | Nafion NR50 | 0.01 | 1 | 181 | 92 |
| Example 10 | Nafion Powder | 0.95 | 0 | 281 | 77 |
| Example 11 | Nafion Powder | 0.95 | 0.12 | 87 | 35 |
| Example 12 | FER (18) | ≦0.93 | 0 | 85 | 36 |
| Comparative | Na-substituted ZSM-5 (51) | 0.23 | 0 | 360 | 75 |
| Example 4 | | | | | |
| Example 12 | Na-substituted USY (10) | 0.12 | 0 | 183 | 88 |

| | | | | Desorption | Desorption |
|---|---|---|---|---|---|
| | | | | of water of | of water of |
| | Polymer | | Hammett's acid | catalyst | catalyst |
| | selectivity | | Strength function | (32-250° C.) | (50-200° C.) |
| | (%) | Color of polymer | H₀ | (%) | (%) |
| Example 1 | 87 | Colorless transparent | +1.5 < Hₒ ≦ +3.3 | 12 | 10 |
| Example 2 | 58 | Yellow | −3 < Hₒ ≦ +1.5 | 8.8 | 7.7 |
| Example 3 | 78 | Colorless transparent | −3 < Hₒ ≦ +1.5 | 7.8 | 6.7 |
| Example 4 | 72 | Pale brown | 6.8 < Hₒ | — | — |
| Comparative | 37 | Brown | −5.6 < Hₒ ≦ −3 | 2.4 | 2.1 |
| Example 1 | | | | | |
| Comparative | 33 | Brown | −5.6 < Hₒ ≦ −3 | — | — |
| Example 2 | | | | | |
| Example 5 | 99 | White | +1.5 < Hₒ ≦ +3.3 | — | — |
| Example 6 | 68 | Brown | −3 < Hₒ ≦ +1.5 | 9.7 | 8.6 |
| Example 7 | 84 | Colorless transparent | +1.5 < Hₒ ≦ +3.3 | — | — |
| Example 8 | 79 | Colorless transparent | −3 < Hₒ ≦ +1.5 | 9.0 | 7.7 |
| Comparative | 47 | Brown | −5.6 < Hₒ ≦ −3 | 2.4 | 2.1 |
| Example 3 | | | | | |
| Example 9 | 98 | White | −3 < Hₒ ≦ +1.5 | — | — |
| Example 10 | 79 | Brown | +1.5 < Hₒ ≦ +3.3 | — | — |
| Example 11 | 84 | Colorless transparent | +1.5 < Hₒ ≦ +3.3 | — | — |
| Example 12 | 72 | Pale yellow | — | 6.2 | 5.5 |
| Comparative | 30 | Brown | — | 2.3 | 1.9 |
| Example 4 | | | | | |
| Example 12 | 75 | Colorless transparent | — | 7.6 | 6.4 |

( ): SiO₂/Al₂O₃ molar ratio
*: Molar ratio to acid amount

Although the present invention has been described in detail and by reference to the specific embodiments, it is apparent to one skilled in the art that various modifications or changes can be made without departing the spirit and scope of the present invention.

This application is based on Japanese Patent Application No. 2004-191567 filed Jun. 29, 2004, Japanese Patent Application No. 2004-191568 filed Jun. 29, 2004, Japanese Patent Application No. 2004-242744 filed Aug. 23, 2004, and Japanese Patent Application No. 2004-242745 filed Aug. 23, 2004, the disclosures of which are incorporated herein by reference in their entities.

### INDUSTRIAL APPLICABILITY

According to the present invention, a polyether polyol having less coloration can be obtained with good selectivity in high yield by dehydrocondensing a polyol using a solid catalyst.

1: A method of producing a polyether polyol, wherein in producing a polyether polyol by dehydrocondensation reaction of a polyol, a solid acid catalyst satisfying at least one of the following requirements (1) to (3) is used:
acid function H0 measured by Hammett's indicator adsorption method is larger than −3;
in Temperature-Programmed Desorption (TPD) analysis of ammonia, desorption amount of ammonia in a region of from 100 to 350° C. is 60 or more of the entire ammonia desorption amount (a region of from 25 to 700° C.); and
in thermogravimetry (TG), desorption amount of water is 3% by weight or more of a reference weight in a region of from 32 to 250° C.

2: The method of producing a polyether polyol as claimed in claim 1, wherein the solid acid catalyst contains a metal element and/or an organic base in an amount of from 0.01 to 2.5 equivalents to the acid.

3: The method of producing a polyether polyol as claimed in claim 2, wherein the metal element is an alkali metal.

4: The method of producing a polyether polyol as claimed in claim 2, where in the organic base has a pyridine skeleton.

5: The method of producing a polyether polyol as claimed in claim 1, wherein the solid acid catalyst, and a metal element-containing compound and/or an organic base are used in combination.

6: The method of producing a polyether polyol as claimed in claim 1, wherein the solid acid catalyst is at least one selected from the group consisting of an intercalation compound, zeolite, a mesoporous substance, a metal composite oxide, an oxide or a composite oxide containing a sulfonic acid group, a carbon material containing a sulfonic acid group, and a resin having a perfluoroalkylsulfonic acid group at a side chain.

7: The method of producing a polyether polyol as claimed in claim 1, wherein the polyol is a diol of from 3 to 10 carbon atoms having two primary hydroxyl groups (excluding a diol that forms a 5-membered or 6-membered cyclic ether by dehydration), or a mixture of the diol and other polyol, wherein the proportion of the other polyol is less than 50 mol %.

8: The method of producing a polyether polyol as claimed in claim 1, wherein the dehydrocondensation reaction is conducted at a temperature of from 120 to 250° C.
